(19) United States
(12) Patent Application Publication
(10) Pub. No.: US 2024/0090511 A1
(43) Pub. Date: Mar. 21, 2024

(54) Modified Insecticidal Proteins

(71) Applicants: BASF Corporation, Florham Park, NJ (US); University of Florida Research Foundation Incorporated, Gainesville, FL (US)

(72) Inventors: Razvan Dumitru, Chapel Hill, NC (US); Bryony C. Bonning, Gainsville, FL (US)

(73) Assignees: BASF Corporation, Florham Park, NJ (US); University of Florida Research Foundation Incorporated, Gainesville, FL (US)

(21) Appl. No.: 18/253,010

(22) PCT Filed: Nov. 15, 2021
(86) PCT No.: PCT/US2021/059370

§ 371 Date: May 15, 2023

Related U.S. Application Data
(60) Provisional application No. 63/114,278, filed on Nov. 16, 2020.

(51) Int. Cl. A01N 63/50; A01P 7/04; C12N 15/82

(52) U.S. Cl. CPC A01N 63/50; A01P 7/04; C12N 15/8286

(57) ABSTRACT

Provided herein are modified insecticidal proteins comprising an insecticidal protein, wherein the insecticidal protein has been modified to include at least one stink bug gut binding peptide.

### CROSS-REFERENCE TO RELATED APPLICATIONS

This application claims the benefit of priority to U.S. Provisional Application No. 63/114,278, filed Nov. 16, 2020, the disclosure of which is incorporated herein by reference in its entirety.

### BACKGROUND

Stinkbugs (Hemiptera; Pentatomidae) are among the most important pests of global agriculture. These piercing-sucking insects impact 12 major agricultural crops across the globe 1, including cotton, soybean and maize^{2,3}. Southern green stink bug (Nezara viridula) along with several other species in the complex constitute a considerable threat to agricultural productivity^{4,5}. The polyphagous habit of N. viridula^{4,6}, makes management of this species particularly problematic. As current stink bug management relies heavily on the application of chemical insecticides that lack target specificity, there remains a need in the art for the development of novel, environmentally friendly insecticide approaches

### INCORPORATED BY REFERENCE OF INFORMATION SUBMITTED ELECTRONICALLY

This application contains, as a separate part of the disclosure, a Sequence Listing in computer readable form (Filename: 202989_Seqlisting.txt; Size: 50,669 Bytes; Created: Nov. 15, 2021), which is incorporated by reference in its entirety.

### SUMMARY

In one aspect, described herein is a chimeric Nezara insecticidal protein comprising a toxic portion and at least one Nezara gut binding protein portion.

### BRIEF DESCRIPTION OF THE FIGURES

FIG. 1A is a gel showing that NvBP1 binds α-amylase N4. 2D gel electrophoresis of 50 μg BBMV, followed by ligand blot analysis with NvBP1-(AP)5-mCherry or (AP)5-mCherry (negative control) as ligand resulted in identification of a single protein spot. Representative ligand blots and a silver stained 2D gel are shown. M. molecular mass markers.

FIG. 1B is a gel showing peptide binding to N. viridula BBMV. Pull-down assays were conducted to assess the relative binding of peptide-(AP)5-mCherry (10 nM) to BBMV (10 μg). Western blots are shown beneath histograms of quantified band intensity for NvBP1 and NvBP5, and for ABP5. For ABP5, binding to BBMV was outcompeted by excess biotinylated synthetic peptide (1-1000 μM) indicating that ABP5 binding is specific. Negative controls used in these assays were (AP)5-mCherry (linker-mCherry), mCherry, and BBMV.

FIG. 2 depicts the modification of ARP147 with Nezara viridula gut binding peptides. The protein structure generated by PyMOL (The PyMOL Molecular Graphics System, Version 2.2 Schrödinger, LLC) with sites of modification indicated by arrows are shown. The protein was modified either by addition of peptide sequences to existing amino acid sequence, or by substitution of existing amino acids. Modifications made with NvBP1 and ABP5 (a subset of sites modified with NvBP1) are shown. See accompanying table for specific details of peptide addition to ARP147. The 21 sites in ARP147 used for peptide addition are denoted in the construct name. A single amino acid (e.g. AA8) indicates the ARP147 amino acid after which the 7 amino acid peptide was added. A peptide range (e.g. AA19-25) indicates the ARP147 amino acids that were replaced. The amino acid sites highlighted in red were used for modification with both NvBP1 and ABP5 peptides separately.

FIG. 3A provides graphs showing the relative binding of peptide-modified ARP147 to N. viridula BBMV. Pull-down assays were performed with native and peptide modified ARP147-MBP. Relative binding of ARP147-MBP modified with NvBP1 or ABP5 is indicated by quantification of western blot band intensities. Data are representative of two biological replicate experiments.

FIG. 3B provides graphs showing the binding affinity of peptide-modified ARP147-MBP to N. viridula BBMV and recombinant APN. Micro-scale thermophoresis was performed for assessment of binding affinity (Kd). In these assays, 25 nM of the target (NHS RED 2nd generation kit dye labeled modified ARP147-MBP) was titrated with 16 serial dilutions of ligand. Ligands were BBMV for NvBP1-modified constructs, and recombinant APN for ABP5-modified constructs. Ligand concentrations ranged from 0.5 μM to 5e-11 pM. The Fnorm value (Base line corrected fluorescence value) is plotted on the Y-axis and ligand concentration on the X-axis. The Kd value is determined by the concentration of the ligand at which 50% of the target is bound to the ligand. The mean Kd values with binding curves are shown for NvBP1- and ABP5-modified ARP147-MBP.

FIG. 4 provides graphs showing that gut binding peptides NvBP1 and ABP5 increased ARP147-MBP toxicity against N. viridula nymphs. Membrane feeding assays with 1 mg/ml ARP147-MBP in Lygus hesperus diet were conducted with 20 second instar N. viridula per biological replicate and four biological replicates. Mortality was recorded every 24 hours for 7 days. Statistically significant differences relative to ARP-147-MBP are indicated by *, ** and ***, representing p<0.05, 0.005 or 0.0005 respectively.

### DETAILED DESCRIPTION

The present disclosure is based on the discovery that modification of an insecticidal protein to include a gut binding peptide enhances the efficacy of the insecticidal protein against stink bugs. As shown in the Examples, the ETX/Mtx2—type protein ARP147, we conclude that: 1) peptides selected for binding to BBMV or to a specific gut surface protein were both effective for enhancing the binding of an ETX/Mtx2 pesticidal protein to the gut of N. viridula, 2) the strength of binding of peptide-modified fusion proteins did not correlate directly with insecticidal activity, 3) in terms of improving insecticidal efficacy, substitution of amino acids in a given ETX/Mtx2 pesticidal protein with gut binding peptide sequences was more successful than addition of peptide sequence to existing sequence. The use of ARP147-MBP fusion proteins for this work facilitated both expression and stability of the ARP147 native and modified toxins. Minimal impact of MBP on any of the parameters tested was observed. MBP would not be included on expression of modified ARP147 in transgenic plants however, reflecting a very different environment for protein expression.

Multiple methods have been adopted for screening phage libraries for peptides that bind to the surface of insect guts with the goal of blocking pathogen transmission^{44, 45} or enhancement of pesticidal proteins^{33, 41} These methods include feeding the target insect on the phage library and eluting bound phage from the dissected gut (e.g. pea aphid, mosquito), the use of BBMV enriched for gut surface proteins, and the use of specific recombinant gut proteins as bait for the phage display library as described herein. For tractable insects, elution from the dissected gut provides the most direct approach for isolation of gut binding peptides without the potential for binding to intracellular gut proteins that are typically present in BBMV⁴⁶. For N. viridula, BBMV were screened for gut binding peptides followed by confirmation of binding to gut surface proteins to avoid the potential loss of phage on exposure to the diverse proteolytic enzymes present in the N. viridula gut and saliva^{39, 47}.

In contrast to other insects, stink bugs rely on a biphasic digestive process, with serine proteases active at alkaline pH released in the saliva, and cathepsins active at acidic pH prevalent in the gut, to ensure complete digestion of ingested materials^{39, 48}. This efficient digestive physiology suggests that pesticidal proteins that bind gut surface proteins abundant in the anterior midgut (M1 region) are more likely to exert an effect than proteins that would have to withstand prolonged exposure to proteases before binding gut proteins in the more distal M2 or M3 regions. Thus knowledge of the relative abundance of surface proteins along the length of the stink bug gut⁴⁹ provides valuable information for selection of appropriate gut surface proteins to target for production of gut binding peptides. In this study, while APN activity predominates in M1, transcript levels are comparable along the length of the gut, suggesting that inhibitors or post-translational mechanisms are involved in regulating APN activity⁴⁹. In contrast to APN, alpha amylase N4 transcripts are abundant in M1, but not in M2 and M3. Modification of ARP147 with NvBP1, which binds alpha amylase resulted in greater toxicity than modification with ABP5, which binds APN, possibly due to increased contact with the gut epithelium in M1 and relatively short exposure to gut digestive enzymes.

While receptor proteins that mediate toxic action have been identified for a number of Bt-derived pesticidal proteins from different structural groups, none have been identified for ETX/Mtx2 proteins. Some of the three-domain Cry toxins bind the GPI anchored midgut proteins ALP and APN, resulting in pore formation^{38, 53}. APN is a functional receptor in several lepidopteran species^{54, 55} with mutation conferring resistance to three-domain Cry toxins in some pest species⁵⁶. In this study, ARP147-MBP was shown to bind recombinant N. viridula APN with a Kd of ˜175 nM indicating that APN is also a putative receptor for ARP147.

### EXAMPLES

Materials and Methods

Expression and purification of N. viridula APN. The appropriate APN contig from an N. viridula midgut transcript library³⁴ was identified based on the presence of conserved protein domains and a predicted GPI anchor³⁵. Recombinant N. viridula APN was baculovirus expressed using pOET3 and Flashbac Ultra (Oxford Expression Technologies, Oxford, UK) in Sf9 and Sf21 cell monolayer cultures maintained in Sf900 SFMIII growth medium (Life Technologies/Thermo Fisher Scientific, Carlsbad, CA) at 27° C. using standard procedures^{36, 37}. All protein concentrations reported herein were determined by Bradford assay (BioRad, Hercules, CA) using bovine serum albumin as a standard. Recombinant protein samples were analyzed with protein separation (10 μg per lane) in a 10% SDS PAGE gel and proteins transferred to a PVDF membrane (Amersham Life Science, Little Chalfont, UK). The membrane was blocked with 1×PBS 0.2% Tween 20 and 5% non-fat dry milk. Recombinant APN was detected with a V5 epitope polyclonal antibody (Rockland Immunochemicals Inc, Gilbertsville, PA; dilution 1:2,000) and an HRP-coupled secondary antibody (Thermo Fisher Scientific, Carlsbad, CA: dilution 1:5,000) followed by a chemiluminescent substrate (Pierce Thermo Scientific, Rockford, Illinois).

Recombinant APN was purified using a Ni NTA agarose column (Sigma-Aldrich, St Louis, MO) using standard procedures. Aminopeptidase N activity was assayed using L-leucine-p-nitroanilide (LpNA) as substrate. Protein (5 μg) was added to a solution containing 80 μL H₂O, 40 μL 1M Tris-HCl pH 7.0, 50 μL 1M NaCl, 20 μL of 10 mMM LpNA. The reaction was incubated for two hours at 37° C. and read at 405 nm in a spectrophotometer. The specific activity was calculated with one unit of specific APN activity defined as the amount of enzyme catalyzing the hydrolysis of 1 mol of LpNA min⁻¹ mg of protein³⁸.

Phage display library screens for peptides that bind N. viridula BBMV or Aminopeptidase N: The Ph.D.-C7C Phage Display Peptide Library (New England Biolabs, Ipswich, MA) was screened either against N. viridula BBMV or recombinant APN. For preparation of brush border membrane vesicles (BBMV), N. viridula were reared as described previously³⁹. For BBMV derived from newly molted adults, insects were immobilized for half an hour over ice and guts dissected. BBMV were prepared by use of the magnesium precipitation method⁴⁰ and stored at −70° C. until use. BBMV (60 μg/ml in TBS buffer with 0.1% Tween) or recombinant APN (100 μg/ml in TBS buffer with 0.1% Tween) were coated overnight on a rocking platform at 4° C. on to polystyrene microtiter wells. Following incubation with the phage library and 10 washes, bound phages were eluted at acidic pH (2.2) following the recommended screening procedures. Eluted phages were amplified for the next round of biopanning. After the third round of phage enrichment, the phage DNA from single plaque forming units was isolated according to the manufacturer's protocol and sequenced by Sanger sequencing at the Iowa State University DNA Facility. Sequences were analyzed with the Clustal Omega server, with grand average of hydropathy (GRAVY) score estimated and screened for unrelated-target binding using the Scanner And Reporter Of Target-Unrelated Peptides (SAROTUP) server.

Two peptides from the BBMV library screen (NvBP1 and NvBP5) and five peptides from the APN library screen (ABP1-5) were selected. Peptide-linker-mCherry fusions were produced with the linker comprised of five alanine proline repeats (AP)5. Primer sequences are provided in Table 1.

TABLE 1
Primers used for production of NvBP- and ABP- mCherry fusion protein
constructs. Restriction sites are shown in bold, peptide sequences
are underlined, the Alanine -Proline linker is shown
in italics and mCherry sequence is shown in bold italics.

| Peptide | Oligonucleotide sequence (5′->3′) |
|---|---|
| Linker-mCherry | TTT GAA TTC GCA CCA GCC CCT GCA CCA GCC CCT |
| | GCA CCA ATG GTG AGC AAG GGC GAG G (SEQ ID NO: 9) |
| NvBP1 (NAGHLSQ, | TTT GAA TTC AAT GCG GGT CAT CTG TCT CAG GCA |
| SEQ ID NO: 1) | CCA GCC CCT GCA CCA GCC CCT GCA CCA ATG GTG |
| | AGC AAG GGC GAG G SEQ ID NO: 10) |
| NvBP5 (EVMSHKW, | TTT GAA TTC GAG GTG ATG TCT CAT AAG TGG GCA |
| SEQ ID NO: 2) | CCA GCC CCT GCA CCA GCC CCT GCA CCA ATG GTG |
| | AGC AAG GGC GAG G SEQ ID NO: 11) |
| ABP1 (GWTHSHG, | TTT GAA TTC GGT TGG ACG CAT AGT CAT GGT GCA |
| SEQ ID NO: 3) | CCA GCC CCT GCA CCA GCC CCT GCA2CCA ATG GTG |
| | AGC AAG GGC GAG G (SEQ ID NO: 12) |
| ABP2 (TDTKYTM, | TTT GAA TTC ACT GAT ACG AAG TAT ACT ATG GCA |
| SEQ ID NO: 4) | CCA GCC CCT GCA CCA GCC CCT GCA CCA ATG GTG |
| | AGC AAG GGC GAG G (SEQ ID NO: 13) |
| ABP3 (SFGMSKA, | TTT GAA TTC TCG TTT GGT ATG TCG AAG GCG GCA |
| SEQ ID NO: 5) | CCA GCC CCT GCA CCA GCC CCT GCA CCA ATG GTG |
| | AGC AAG GGC GAG G (SEQ ID NO: 14) |
| ABP4 (WPGHSA1, | TTT GAA TTC TGG CCT GGG CAT TCT GCG ATT GCA |
| SEQ ID NO: 6) | CCA GCC CCT GCA CCA GCC CCT GCA CCA ATG GTG |
| | AGC AAG GGC GAG G (SEQ ID NO: 15) |
| ABP5 (MDWPRRN, | TTT GAA TTC ATG GAT TGG CCT AGG CGT AAT GCA |
| SEQ ID NO: 7) | CCA GCC CCT GCA CCA GCC CCT GCA CCA ATG GTG |
| | AGC AAG GGC GAG G (SEQ Id NO: 16) |
| mCherry Reverse | GCT TGG CTG AAG CTT CTT GTA CAG CTC GTC CAT |
| | GCC (SEQ ID NO: 17) |

Peptide-(AP)5-mCherry constructs were cloned into pBAD/HisB (Invitrogen) and proteins expressed and purified as described by Chougule et al. 2013⁴¹.

Identification of the gut protein bound by NvBP1: Two-dimensional ligand blot analysis using N. viridula gut-derived BBMV (50 μg treated with the 2D Clean-up kit; GE Healthcare, Chicago, IL) was used for identification of the NvBP1 gut binding partner as described previously⁴². 2D-separated proteins were transferred from the gel to a PVDF membrane (Millipore, Burlington, MA) for ligand blot, N-terminal sequencing, or LC MS/MS identification. For proteins transferred to PVDF membranes, the membrane was blocked 1 hour with 1×PBS with 0.2% Tween 20 (PBST) and 5% fat free milk. Following five washes with (PBST), the blot was incubated 2 hours with 10 mM of either NvBP1 peptide-mCherry fusion or (AP)5 linker—mCherry fusion in PBST with 0.1% fat free milk. The blots were rinsed and washed five times with PBST and incubated with the primary antibody, anti-mCherry (Novus Biologicals, Littleton, CO) at a 1:5000 dilution for 1 hour. The membrane was washed as described, incubated with the secondary antibody anti-rabbit coupled to HRP (ratio 1:10000, Thermo Scientific) for 1 hour and washed. A final wash with 1×PBS was performed before exposing the blot to film. Antibody binding was detected with the West—Pico Chemioluminescent kit (Pierce/Thermo).

The NvBP1 binding protein was identified by LC MS/MS and N-terminal sequencing. For LC MS/MS protein identification, the protein spot was manually excised from the 2D gel, reduced, alkylated and digested with trypsin using standard procedures. The generated peptides were then separated by LC MS/MS with the Q Exactive™ Hybrid Quadrupole-Orbitrap Mass Spectrometer (Thermo Scientific). The translated Nezara viridula transcriptome 34 was used as reference to identify N. viridula proteins. The TMHMM Server v. 2.0 was used to predict the presence of transmembrane helices in candidate NvBP1 binding proteins.

N-terminal sequencing was performed after 2D gel electrophoresis and transfer of BBMV proteins to a PVDF membrane. N-terminal protein sequencing was carried out on proteins visualized by staining (Coomassie brilliant blue R250) by Edman degradation with a Perkin Elmer Applied Biosystems Model 494 Procise protein/peptide sequencer (Norwalk, CT) with an on-line Perkin Elmer Applied Biosystems Model 140C PTH Amino Acid Analyzer.

Confirmation of peptide binding to BBMV by pull-down assay: To confirm binding of selected peptides to N. viridula gut proteins, pull-down assays were conducted with purified N. viridula binding peptide—linker-mCherry fusions and N. viridula BBMV. Fusion proteins (10 nM) were incubated with 10 μg BBMV in binding solution (1×PBS 0.1% Tween 20 and 0.1% BSA) in a final volume of 100 μL. This solution was incubated for two hours at room temperature and centrifuged at 20,800×g for 20 minutes at 4° C. The pellet was resuspended with 100 μL of binding solution and centrifugation repeated three times. Finally, the resulting pellet was resuspended in 10 μL binding solution and analyzed by western blot. Proteins were resolved in a 10% SDS PAGE gel and transferred to PVDF membrane (Amersham). The membrane was blocked with 1×PBS 0.2% Tween 20 and 5% non-fat dry milk. The peptide fusion was detected using an mCherry polyclonal antibody (Thermo Scientific, dilution 1:5000) and a secondary HRP coupled antibody (Thermo Scientific, dilution 1:5000) followed by a chemiluminescent substrate (Pierce Thermo Scientific). For competition bioassays conducted with peptides ABP1-5, the peptide mCherry fusion proteins were incubated with or without excess peptide (biotinylated, 0.1 μM to 1000 nM) with BBMV derived from second instar nymphs, and the pull-down assay continued as described above.

Peptide modification of ARP147: ARP147 is an ETX/Mtx2 pesticidal protein that shares 24% amino acid identity to the f3 pore-forming E-toxin from Clostridium perfringens. The predicted structure of ARP147 was modeled by I-TAS SER. Sites for introduction of peptides NvBP1 or ABP5 by addition to—or replacement of—existing sequence were selected on the basis of homology modeling, in silico protein stability and peptide exposure on the surface of the protein. Sequences for the modified ARP147 constructs were synthesized by GenScript for expression as HisMBP fusion proteins in pMal-c2X (New England Biolabs). Native and peptide-modified ARP147-MBP fusions were expressed in E. coli BL21 cells and purified (pMal Protein Fusion and Purification System; New England Biolabs). The purified toxins were concentrated using the Amicon Ultra Centrifugal Filter 6 ml device (Millipore Sigma, Burlington, MA).

Assessment of peptide impact on toxin binding: Both pull-down assays and microscale thermophoresis (MST) were used to assess the relative binding of native and modified ARP147-MBP. For the pull-down assay, the protocol described above was followed using 50 nM of the respective ARP147-MBP along with 10 μg of N. viridula BBMV. Fusion proteins that bound BBMV were visualized by western blot with anti-ARP147 antibody raised in rabbit.

For MST analysis, a Monolith NT 115 (NanoTemper, Cambridge, MA) was used to determine the binding affinity between 1) ARP147-MBP modified with NvBP1 and BBMV, and 2) ARP147-MBP modified with ABP5 and recombinant N. viridula APN. The native and modified ARP147-MBP (˜75 kDa) were labeled using the Monolith Protein Labeling Kit RED-NHS 2^{nd} Generation (Amine Reactive) according to the manufacturer's directions and aliquoted in 20 μl or smaller volumes in Corning Costar low binding microcentrifuge tubes. To determine binding affinity, 50 nM of target (labeled ARP147-MBP diluted from a 1 μM stock concentration with MST assay buffer; 1×PBS with 0.1% Tween-20) and 111M of ligand (solubilized BBMV or recombinant APN) were used. A sixteen-fold serial dilution of the ligand was made in low binding tubes using ligand buffer (5% CHAPS buffer). Target (100) was added to the serially diluted ligand (100) and incubated in the dark at room temperature for 10 minutes. The Monolith NT115 series premium capillaries were dipped into the final mix, and arranged in the order of high to low ligand concentrations in the capillary stand, which was then inserted into the Monolith NT115. Readings were taken for the ΔF norm. The normalized fluorescence (Fnorm) for each data point represented by the interaction between fluorescent-labeled target molecule (ARP147-MBP constructs) at a particular concentration with a range of concentrations of unlabeled ligand (BBMV or APN) was plotted into a sigmoid curve to obtain the Kd value, where half of the target molecules are in a bound state. Response amplitude and the signal to noise ratios were used as measures of quality control. Three biological replicates were performed for selected modified toxins.

Bioassays: Toxicity of native and modified ARP147-MBP to N. viridula was assessed using the bioassay method of Wellman-Desbiens and Côté⁴³. Scintillation vials were set up with a layer of autoclaved sand, 100 μl of ddH₂0 followed by another layer of autoclaved sand and plugged with autoclaved cotton plugs. Four stink bugs were placed in each vial. The purified toxins (1 mg in 1 ml for the six selected constructs) were mixed with Lygus hesperus artificial diet (Frontier Scientific Services Inc, Newark, Delaware, US) supplemented with 150 μl of streptomycin (500 μg/ml) and 350 of Nystatin (50 mg/ml) poured into diet packets made with parafilm. The assay was conducted with four biological replicates of twenty 2^{nd} instar nymphs. Insect mortality was recorded daily for 7 days. Statistical differences between modified and wild type constructs were determined by Student's t-test.

### Example 1—Identification of Peptides that Bind BBMV or Recombinant APN of N. viridula

Screening of the Ph.D.-C7C Phage Display Peptide Library for peptides that bound N. viridula BBMV resulted in identification of candidate peptides NAGHLSQ (NvBP1, SEQ ID NO: 1) and EVMSHKW (NvBP5, SEQ ID NO: 5) by Sanger sequencing. Of sixty phages sequenced after the third round of phage enrichment, forty encoded NvBP1 and one expressed NvBP5. As SAROTUP analysis indicated that NvBP5 had polystyrene binder sequence, this peptide was omitted from further analysis.

Aminopeptidase N (APN) was identified from Contig 9840 from the N. viridula transcriptome 34. Recombinant N. viridula APN was expressed in Sf21 cells using the baculovirus expression system, and affinity purified. Five peptides (ABP1-5) were selected following phage display library screening for phage encoding peptides that bind recombinant N. viridula APN (ABP1-5 are set forth in SEQ ID NOs: 3-7, respectively). ABP5 (SEQ ID NO: 7) was the most hydrophilic of the seven peptides assessed.

### Example 2—NvBP1 Binds to N. viridula α-Amylase

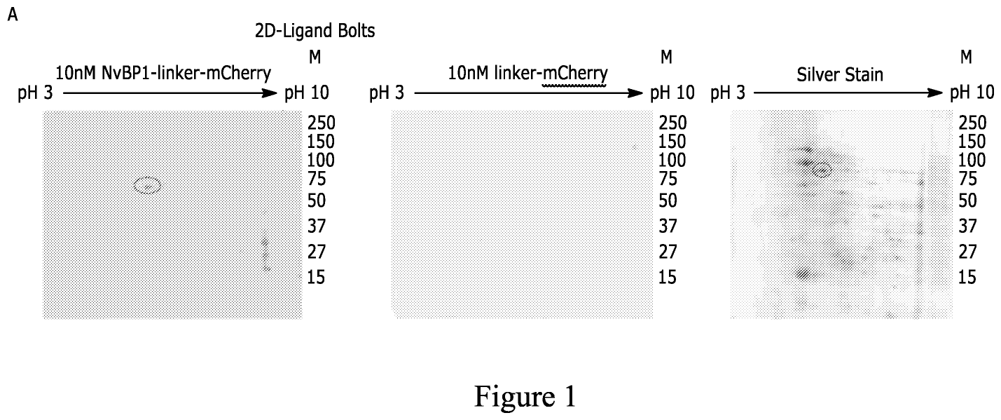

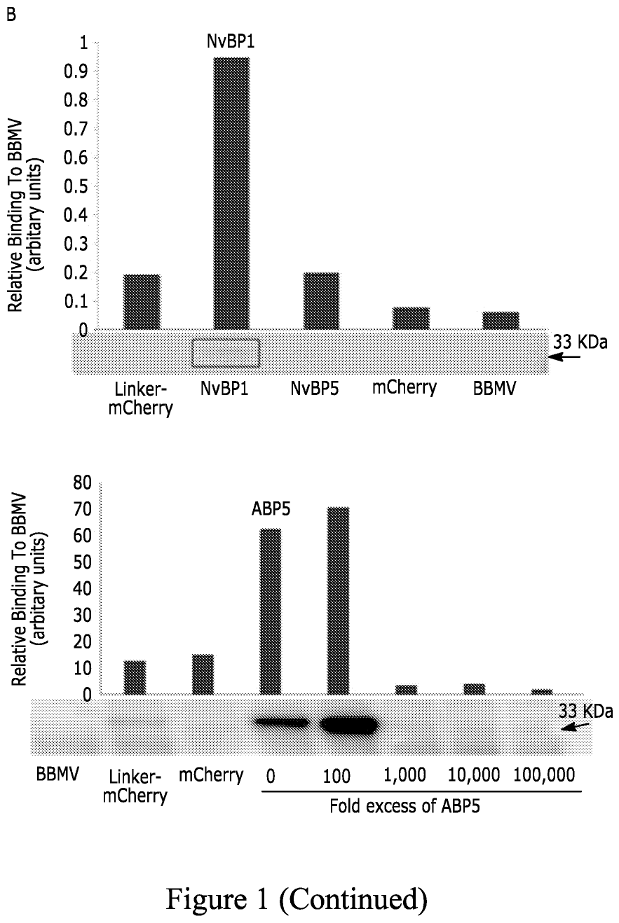

2D ligand blot analysis with NvBP1-(AP)5-mCherry showed that NvBP1-(AP)5-mCherry binds a ˜50 kDa protein with a pI of 6 that was absent from the negative control blot with (AP)5-mCherry as ligand (FIG. 1A). While four candidate binding proteins were identified by LC-MS/MS with reference to the translated N. viridula gut transcriptome, the approximate pI (5.62 and 6.02) and molecular mass of proteins encoded by two contigs, 9247 (56 kDa) and 10931 (60 kDa), correlated with the protein observed in the ligand blot. Alpha-amylase N4 on the surface of the N. viridula gut epithelium is the binding partner of NvBP1 based on localization (transmembrane helix from Y13 to A35) and the N-terminal sequence of DTIXN (SEQ ID NO: 8). Ligand blot results also indicate that the binding of NvBP1 is specific.

### Example 3—Peptide Binding and Specificity of Binding to BBMV Proteins

Pull-down assays conducted to confirm binding of peptides selected from the phage display library screens showed that NvBP1, but not NvBP5 bound to N. viridula BBMV proteins (FIG. 1B). This result is consistent with characterization of NvBP5 as a polystyrene binding peptide.

While binding of all five APN binding peptide-(AP)5-mCherry fusions (ABP1-5) to BBMV was confirmed (data not shown), only ABP5-(AP)5-mCherry bound specifically to BBMV with binding outcompeted at >10 μM peptide in competition pull-down assays (FIG. 1B). On the basis of these competition assays, peptide ABP5 (SEQ ID NO: 7) was selected for modification of ARP-147, along with NvBP1 (SEQ ID NO: 1).

### Example 4—Peptide Addition to ARP147-MBP

A homology model based on Mpp51Aa1 was used for selection of sites for peptide modification. As there was little information on domains of ETX/Mtx2 proteins that are important for toxicity, a wide range of sites including alpha helices, beta sheets and loop regions that are predicted to be on the exterior of ARP147 were selected for modification. The sites and the mode of peptide addition (addition to—or substitution of—existing sequence), were selected on the basis of modeling with 1) the peptide predicted to be displayed on the surface of ARP147 rather than folded in, and 2) the stability of the predicted modified structure.

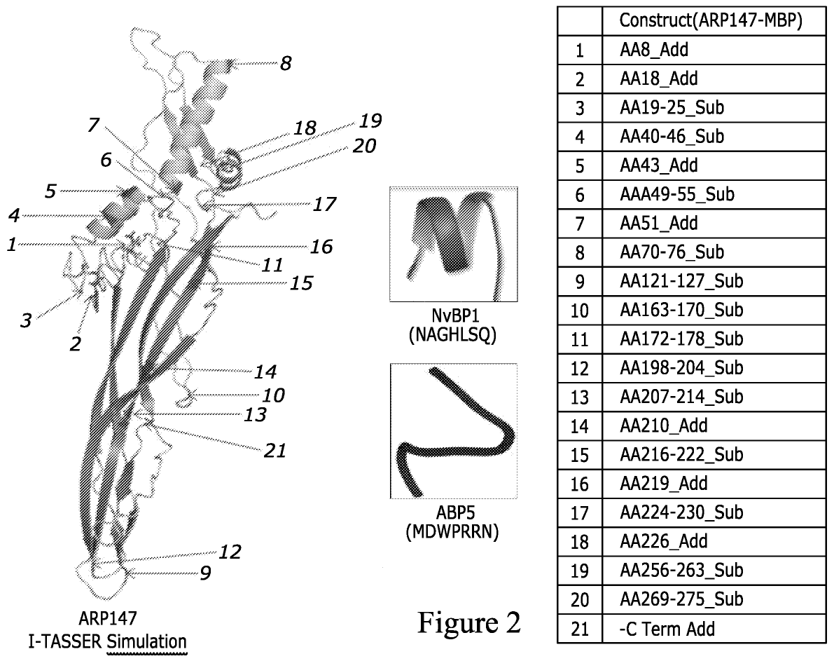

NvBP1 was incorporated into eight sites in ARP147 by addition- and into 13 sites by substitution—of existing amino acid sequences, resulting in a total of 21 constructs (FIG. 2). All 21 NvBP1-modified ARP147-MBP expressed stably in E. coli (data not shown). Based on data generated from initial bioassays with all 21 constructs (not shown), a subset of six NvBP1-modified constructs was selected for further analysis. These same six sites (one addition at AA43 (SEQ ID NOs: 25, 27 and 29), five substitution; AA70-76 (SEQ ID NOs: 31, 33, 35), AA172-178 (SEQ ID NO: 39), AA207-214 (SEQ ID NOs: 19, 21 and 23), AA224-230 (SEQ ID NO: 41) and AA269-275 (SEQ ID NO: 37) were also used for ARP147 modification with ABP5 (FIG. 2), and ABP5-modified ARP147-MBP were also stably expressed (data not shown).

### Example 5—Binding of Modified ARP147-MBP to N. viridula Gut Proteins

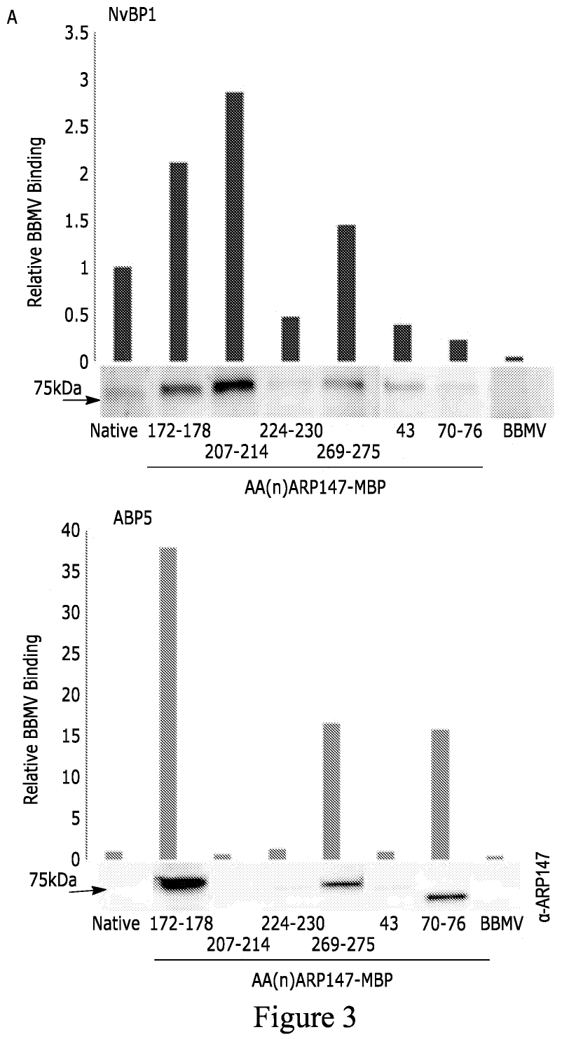

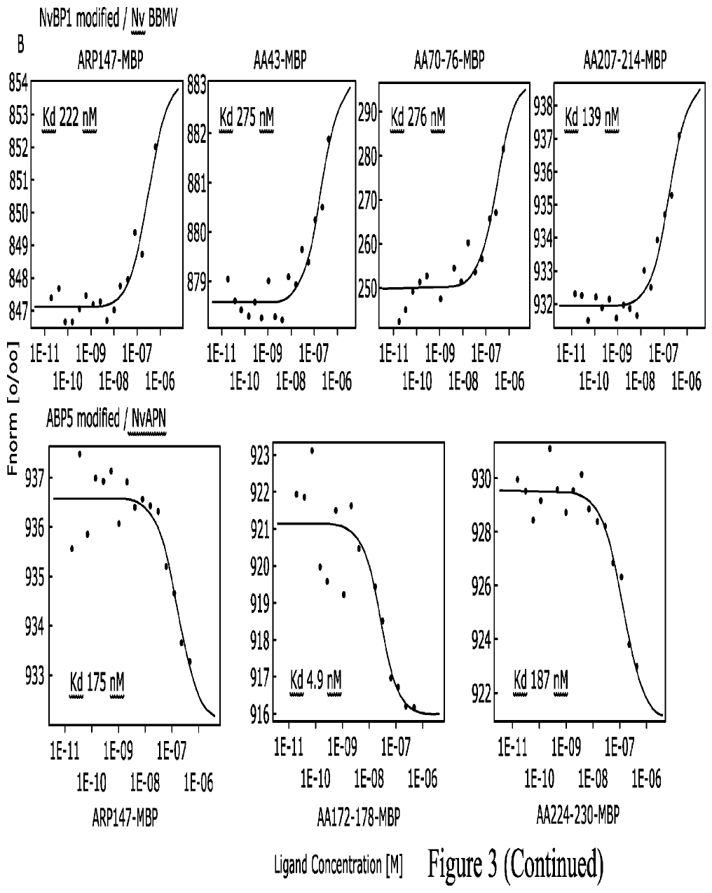

The binding of the 12 modified ARP147-MBP to BBMV was first assessed by pull-down assay. NvBP1-modified constructs with modifications at AA172-178, 207-214 and 269-275 showed increased binding relative to native, with the strongest binding for NvBP1 substitution of AA207-214 (FIG. 3A). NvBP1-modified constructs with modifications at AA224-230, 43 and 70-76 showed decreased binding relative to native in these pull-down assays. ABP5-modified constructs with modifications at sites AA70-76, 172-178 and 269-275 showed increased binding relative to native, with modification at AA172-178 showing the strongest binding. No significant change in binding relative to native ARP147-MBP was seen for the other three ABP5-modified proteins. Taken together, sites AA 172-178 and AA 269-275 resulted in increased binding for both peptides in pull-down assays, while sites AA 207-214 and AA70-76 resulted in increased binding for a single peptide, NvBP1 and ABP5, respectively.

The binding affinity (Kd) values for binding of selected ARP147-MBP modified with NvBP1 or ABP5 to BBMV or recombinant N. viridula APN, respectively were determined by MST. The average Kd for NvBP1-modified ARP147-MBP AA207-214 was 139 nM compared to 222 nM for unmodified, indicating increased binding of this modified protein to BBMV (FIG. 3B). In contrast, binding of NvBP1 constructs 43 and 70-76 decreased relative to native consistent with pull-down assay results. The binding affinity of ABP5-modified ARP147-MBP to recombinant APN increased for AA172-178 (Kd 4.9 nM) while binding of AA 224-230 was comparable to that of native (Kd 175 nM; FIG. 3B). These results are consistent with pull-down assay data. Binding of ARP147-MBP to recombinant N. viridula APN indicates that APN is a putative receptor for this pesticidal protein.

### Example 6—Impact of Peptide-Modified ARP147-MBP on N. viridula

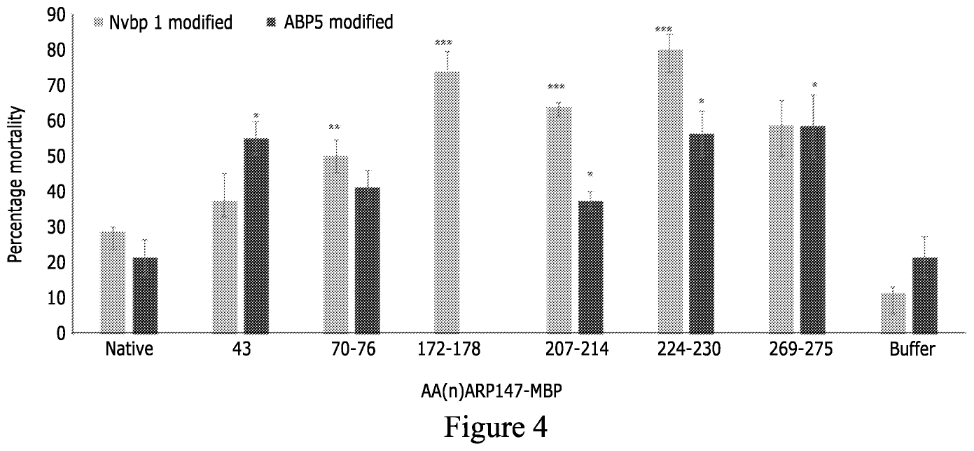

Bioassays were conducted to assess the impact of the twelve peptide-modified ARP147-MBP on second instar N. viridula nymphs. Toxicity was significantly increased for four NvBP1-modified constructs: AA70-76, 172-178, 207-214, and 224-230 (FIG. 4). For ABP5-modified constructs, toxicity was significantly enhanced for the following four constructs: AA43 (SEQ ID NOs: 25, 27 and 29), 207-214 (SEQ ID NOs: 19, 21 and 23), and 224-230 (SEQ ID NO: 41) and 269-275 (SEQ ID NO: 37) (FIG. 4).

Discussion:

Addition of peptides NvBP1 and ABP5 to ARP147 provided artificial anchors for binding to α-amylase N4 and APN, respectively. Alpha-amylase is a receptor for B.t. israelensis mosquitocidal proteins Cry4Ba and Cry11Aa in Anopheles albimanus with binding observed between these pesticidal proteins and recombinant E. coli—expressed alpha amylase⁵⁷. The peptides NvBP1 and ABP5 therefore either increased ARP147-MBP binding to existing receptor proteins (i.e. putative receptor APN), or provided new binding sites (alpha amylase N4) to expedite toxic action. The extent of increased binding and toxic action varied at a given site according to peptide in some cases. At site AA43 for example, the only site for which peptide sequences were added to ARP147 sequence, significant toxicity enhancement was seen with ABP5 but not with NvBP1. For site 70-76, significantly increased mortality was seen for NvBP1 but not ABP5. In these cases, the impact of the peptide sequence on ARP147 structure, or the orientation of ARP147 on binding may drive these different outcomes.

Overall, higher mortality levels were seen for constructs modified with NvBP1 than ABP5, particularly for modifications at AA 172-178, AA 207-214 and AA 224-230. Non mutually exclusive scenarios that may account for this observation include 1) NvBP1 mediates higher affinity binding to α-amylase than ABP5 binding to APN, 2) α-amylase is more abundant in the gut resulting in higher toxicity, 3) α-amylase expression is high in the anterior midgut (M1) relative to APN, such that toxicity occurs before significant enzymatic degradation of ARP147-MBP, as discussed above. ARP147-MBP shows minimal degradation when treated with N. viridula saliva (FIG. S8).

The beta pore forming toxins have a highly variable head region that is hypothesized to interact with receptors in the host gut, and a highly conserved tail region proposed to function in pore formation and membrane integration^{58, 59}. An essential role has been proposed for the beta barrels in ETX/Mtx22 proteins in the formation of pores in their target insects⁶⁰. The six sites employed for modification of ARP147 were scattered throughout the head domain. It is notable that placement of gut binding peptides at AA207-214 in the beta barrel domain did not interfere with toxicity.

The extent of binding did not correlate with enhanced toxicity. For example, ABP5 modification at AA70-76 with increased binding but no enhanced toxicity, and NvBP1 modification at AA224-230 with increased toxicity but no enhanced binding relative to ARP147-MBP. The lack of association between binding and toxicity likely reflects additional factors involved in the mode of toxic action as noted previously⁴¹.

It is contemplated that NvBP1 and ABP5 to bind to species closely related to N. viridula (i.e. other stink bugs). The extent of peptide binding is expected to reflect the similarity of receptor proteins, particularly at the specific peptide binding region. Importantly, none of the N. viridula gut binding peptides identified herein were enriched from the same phage library screened in vivo for peptides that bind to the gut epithelium of the honey bee, Apis mellifera. Although empirical tests are required to test for toxicity to nontarget organisms, the peptides used in this study are not expected to bind the honey bee gut.

In summary, peptides ABP5 (SEQ ID NO: 7) and NvBP1 (SEQ ID NO: 1) were successfully used to enhance the toxicity of an ETX/Mtx2 pesticidal protein against N. viridula nymphs.

### REFERENCES

1. Lavore et al., Scientific Reports 8:17244 (2018).
2. Athey et al., Plos One 14(3): e0214325 (2019).
3. McPherson et al., Stink Bugs of Economic Importance in America North of Mexico; Boca Raton, FL, USA: CRC Press; 2000. 253p p.
4. Depieri et al., Neotropical Entomology 40(2):197-203 (2011).
5. Lomate et al., Scientific Reports 6:27587 (2016).
6. Panizzi R A., American Entomologist 61(4):223-33 (2015).
7. van Frankenhuyzen K., J Invertebr Pathol 114(1):76-85 (2013).
8. Bravo et al., Insect Biochemistry and Molecular Biology 41(7):423-31 (2011).
9. Chen et al., Biochemical Journal 424(1):191-200 (2009).
10. Dorsch et al., Insect Biochemistry and Molecular Biology 32(9):1025-36 (2002).
11. Fabrick et al., Journal of Biological Chemistry 284(27):1807-18410 (2009).
12. Atsumi et al., PNAS, USA, E1591-E1598 (2012).
13. Tanaka et al., FEBS J 280(280(8)):1782-94 (2013).
14. Banerjee et al., Scientific Reports 7(1):10877 (2017).
15. Gill et al., Insect Molecular Biology 11(6):619-25 (2002).
16. Knight et al., Molecular Microbiology 11(3):429-36 (1994).
17. Rajagopal et al., Journal of Biological Chemistry 277(49):46849-51 (2002).
18. Arenas et al., Journal of Biological Chemistry 285(17):12497-503 (2010).
19. Jurat-Fuentes et al., European Journal of Biochemistry 271(15):3127-35 (2004).
20. Fernandez-Luna et al., Environ Microbiol 12(3):746-57 (2010).
21. Ochoa-Campuzano et al., Biochem Bioph Res Co 362(2):427-47 (2007).
22. Griffitts et al., Science 307(5711):922-5 (2005).
23. Chougule et al., Toxins 4:405-29 (2012).
24. Porcar et al., Appl Environ Microbiol 75(14):4897-900 (2009).
25. Crickmore et al., J Invertebr Pathol: 107438 (2020).
26. Baum et al., Journal of Econ Entomol 105(2):616-24 (2012).
27. Gowda et al., Nature Communications 7:12213 (2016).
28. Liu et al., Applied and environmental microbiology 84(3):e01996-17 (2018).
29. Farmer et al., Regulatory Toxicology and Pharmacology 89:155-64 (2017).
30. Bachman et al., PLOS ONE 12(1):e0169409 (2017).
31. Moar et al., Journal of Invertebrate Pathology 142:50-9 (2017).
32. Chougule et al., of the National Academy of Sciences 11(21):8465-70 (2013).
33. Shao et al., Scientific Reports 6:20106 (2016).
34. Liu et al., Insect Biochem Mol Biol 103:36-45 (2018).
35. Eisenhaber et al., Trends Biochem Sci 25(7):340-1 (2000).
36. Hitchman et al., Cell Biol Toxicol 26(1):57-68 (2010).
37. King L A and Possee R D. The Baculovirus Expression System. London: Chapman and Hall; 1992. 229 p.
38. Bravo et al., Biochimica et Biophysica Acta (BBA)—Biomembranes 1667(1):38-46 (2004).
39. Lomate et al., Sci Rep 6:27587 (2016).
40. Wolfersberger M G., Archives of insect biochemistry and physiology 24(3):139-47 (1993).
41. Chougule et al. Retargeting of the Bacillus thuringiensis toxin Cyt2Aa against hemipteran insect pests. (1091-6490 (Electronic)).
42. Linz et al., Virol 89(22):11203-12 (2015).
43. Wellman-Desbiens et al., J. Econ Entomol 98(5):1469-79 (2005).
44. Liu et al., Virology 401(1):107-16 (2010).
45. Ghosh et al., Proc Natl Acad Sci USA 98:13278-81 (2001).
46. Bayyareddy et al., Journal of proteome research 11(12):5843-55 (2012).
47. Canton et al., Front Physiol 10:1553 (2019).
48. Canton et al., Curr Opin Insect Sci 41:86-91 (2020).
49. Canton et al., J Insect Physiol 119:103965 (2019).
50. Arenas et al., The Journal of biological chemistry 285(17):12497-503 (2010).
51. Gilliland et al., and environmental microbiology 68(4):1509 (2002).
52. Lorence et al., Aminopeptidase dependent pore formation of Bacillus thuringiensis Cry1Ac toxin on Trichoplusia ni membranes. (0014-5793 (Print)).
53. Pardo-Lopez et al., Structural changes of the Cry1Ac oligomeric pre-pore from Bacillus thuringiensis induced by N-acetylgalactosamine facilitates toxin membrane insertion. (0006-2960 (Print)).
54. Gill et al., Identification, isolation, and cloning of a Bacillus thuringiensis Cry1Ac toxin-binding protein from the midgut of the lepidopteran insect Heliothis virescens. (0021-9258 (Print)).
55. Knight et al., cloning of an insect aminopeptidase N that serves as a receptor for Bacillus thuringiensis CryIA(c) toxin. (0021-9258 (Print)).
56. Heckel D G. How do toxins from Bacillus thuringiensis kill insects? An evolutionary perspective. Archives of insect biochemistry and physiology 104(2): e21673 (2020).
57. Fernandez-Luna et al., Environ Microbiol 12(3):746-57 (2010).
58. Jerga et al. Mechanistic insights into the first Lygus-active beta-pore forming protein. (1096-0384 (Electronic)).
59. James et al., Journal of Economic Entomology 105(2):616-24 (2012).
60. Popoff M R. Epsilon toxin: a fascinating pore-forming toxin. (1742-4658 (Electronic)).
61. Xu et al. Crystal structure of Cry51Aa1: A potential novel insecticidal aerolysin-type beta-pore-forming toxin from Bacillus thuringiensis. (1090-2104 (Electronic)).
62. Gassmann et al., Proceedings of the National Academy of Sciences 111(14):5141 (2014).
63. Blanco et al., Journal of Economic Entomology 102(1):381-7 (2009).
64. Chougule N P and Bonning B C. Toxins for transgenic resistance to hemipteran pests. (2072-6651 (Electronic)

What is claimed is:

1. A modified insecticidal protein comprising an insecticidal protein, wherein the insecticidal protein has been modified to include at least one stink bug gut binding peptide.

2. The modified insecticidal protein of claim 1, wherein the stink bug gut binding peptide is a Nezara gut binding peptide

3. The modified insecticidal protein of claim 1, wherein the insecticidal protein is a bacterial insecticidal protein.

4. The modified insecticidal protein of claim 3, wherein the insecticidal protein is Cry1A, Cry1B, Cry1C, Cry1D, Cry1E, Cry1F, Cr1I; Cry2, Cry2A family; Cry9, Cry9A, Cry9B, Cry9C, Cry9D, Cry9E, and Cry9F families; or Vip3.

5. The modified protein of claim 1, wherein the stink bug gut binding peptide comprises an amino acid sequence at least 95% identical to the amino acid sequences set forth in any one of SEQ ID NOs: 1-7.

6. The modified protein of claim 5, wherein the stink bug gut binding peptide comprises an amino acid sequence set forth in any one of SEQ ID NOs: 1-7.

7. The modified protein of claim 5, wherein the gut binding peptide comprises the amino acid sequence set forth in SEQ ID NO: 7.

8. The modified protein of claim 3, wherein the insecticidal protein is modified by addition to include the gut binding peptide.

9. The modified protein of claim 3, wherein the insecticidal protein is modified by substitution to include the gut binding peptide.

10. The protein of claim 8, that is construct 1, 2, 5, 7, 14, 16, 18, or 21 set forth in FIG. 2.

11. The protein of claim 9, that is construct 3, 4, 6, 8-13, 15, 17, 19 or 20 set forth in FIG. 2.

12. The protein of claim 4, comprising an amino acid sequence at least 95% identical to an amino acid sequence set forth in any one of SEQ ID NOs: 19, 21, 23, 25, 27, 29, 31, 33, 35, 37, 39 or 41.

13. The protein of claim 4, comprising an amino acid sequence set forth in SEQ ID NOs: 19, 21, 23, 25, 27, 29, 31, 33, 35, 37, 39 or 41.

14. A nucleic acid encoding the modified protein of claim 12.

15. The nucleic acid of claim 14, comprising a nucleotide sequence at least 95% identical to a nucleotide sequence set forth in any one of SEQ ID NOs: 18, 20, 22, 24, 26, 28, 30, 32, 34, 36, 38, or 40.

16. The nucleic acid of claim 14, comprising a nucleotide sequence set forth in any one of SEQ ID NOs: 18, 20, 22, 24, 26, 28, 30, 32, 34, 36, 38, or 40.

17. A host cell comprising the nucleic acid of claim 12.

18. The host cell of claim 17, wherein the host cell is a plant cell.

19. An expression construct comprising a nucleic sequence of claim 12 operably linked to a promoter.

20. A transformed plant comprising the construct of claim 19.

21. A method of providing pesticidal activity in a plant comprising introducing the expression construct of claim 19 into a host cell of the plant and expressing a polynucleotide of claim 14 in said host cell, thereby providing pesticidal activity in the plant.

22. A method of inhibiting plant damage from a stink bug, the method comprising providing a plant expressing the modified protein of claim 5, and allowing a stink bug to feed on the plant, wherein the stink bug is killed and the plant damage is inhibited.


SEQUENCE LISTING (41 sequences)

SEQ ID NO: 1 - protein, 7 aa, N. viridula
NAGHLSQ

SEQ ID NO: 2 - protein, 7 aa, N. viridula
EVMSHKW

SEQ ID NO: 3 - protein, 7 aa, N. viridula
GWTHSHG

SEQ ID NO: 4 - protein, 7 aa, N. viridula
TDTKYTM

SEQ ID NO: 5 - protein, 7 aa, N. viridula
SFGMSKA

SEQ ID NO: 6 - protein, 7 aa, N. viridula
WPGHSAI

SEQ ID NO: 7 - protein, 7 aa, N. viridula
MDWPRRN

SEQ ID NO: 8 - protein, 5 aa, Artificial Sequence
  Synthetic
  misc_feature (4)..(4): Xaa can be any naturally occurring amino acid
DTIXN

SEQ ID NO: 9 - DNA, 58 bp, Artificial Sequence
  Synthetic
tttgaattcgcaccagcccctgcaccagcccctgcaccaatggtgagcaagggcgagg

SEQ ID NO: 10 - DNA, 79 bp, Artificial Sequence
  Synthetic
tttgaattcaatgcgggtcatctgtctcaggcaccagcccctgcaccagcccctgcaccaatggtgagcaagggcgagg

SEQ ID NO: 11 - DNA, 79 bp, Artificial Sequence
  Synthetic
tttgaattcgaggtgatgtctcataagtgggcaccagcccctgcaccagcccctgcaccaatggtgagcaagggcgagg

SEQ ID NO: 12 - DNA, 79 bp, Artificial Sequence
  Synthetic
tttgaattcggttggacgcatagtcatggtgcaccagcccctgcaccagcccctgcaccaatggtgagcaagggcgagg

SEQ ID NO: 13 - DNA, 79 bp, Artificial Sequence
  Synthetic
tttgaattcactgatacgaagtatactatggcaccagcccctgcaccagcccctgcaccaatggtgagcaagggcgagg

SEQ ID NO: 14 - DNA, 79 bp, Artificial Sequence
  Synthetic
tttgaattctcgtttggtatgtcgaaggcggcaccagcccctgcaccagcccctgcaccaatggtgagcaagggcgagg

SEQ ID NO: 15 - DNA, 79 bp, Artificial Sequence
  Synthetic
tttgaattctggcctgggcattctgcgattgcaccagcccctgcaccagcccctgcaccaatggtgagcaagggcgagg

SEQ ID NO: 16 - DNA, 79 bp, Artificial Sequence
  Synthetic
tttgaattcatggattggcctaggcgtaatgcaccagcccctgcaccagcccctgcaccaatggtgagcaagggcgagg

SEQ ID NO: 17 - DNA, 36 bp, Artificial Sequence
  Synthetic
gcttggctgaagcttcttgtacagctcgtccatgcc

SEQ ID NO: 18 - DNA, 888 bp, Artificial Sequence
  Synthetic
atggcggagattgaagaaaacaagaactccacaactgctctttcggaatcaagagaagagctctttggaattgttgatgtcgacaaaagtttcgacaagatggcccagtattactacttcaagaatctccaagggaagatagagaacgggtacaaattcacgaactacaagggaagctcaacgaatctcgataagttcaaaacgacctccgataaagtctctgacttgcaaataaccggcttgcttcctgaatatattgggggaaacactttcccaaatcataccgagcaagagcaaaccttttcaaccacaagctacgcacgtacaataactgagagtgtcacgacaacgactcaaagcggattttctgtttctggacaaggaggattgttgaacatccccatcatttttccaaacggaacctcaatcactgcttcaatcaacacttcctcgactttggcaaagcaagagagtgatacaaggactttaactgcaccttctcaagctgtgaaggttccacctggaaaaacatatcgagctgaagtgaagctggtgagaaaatccttcaaagcgaatgtgggattcaatggaacaattgctcctcccctttcaactgacatatgcaatgctggccatctctctcaatgcggagctggaggaaggccaaataaaactgtcacaaagagctatgccacacaaactctttatcgggatatgaccccttctcaacaaaaagatttggggatcaccttcaactcaacaactggccaatttgacgtgaatgggaaagccgttattgatggaattgctggaagcgaaatggaagtcgcaatctacgatattaccaatgctcgaactccgaagctcgtttcgaagacctattactaa

SEQ ID NO: 19 - protein, 295 aa, Artificial Sequence
  Synthetic
MAEIEENKNSTTALSESREELFGIVDVDKSFDKMAQYYYFKNLQGKIENGYKFTNYKGSSTNLDKFKTTSDKVSDLQITGLLPEYIGGNTFPNHTEQEQTFSTTSYARTITESVTTTTQSGFSVSGQGGLLNIPIIFPNGTSITASINTSSTLAKQESDTRTLTAPSQAVKVPPGKTYRAEVKLVRKSFKANVGFNGTIAPPLSTDICNAGHLSQCGAGGRPNKTVTKSYATQTLYRDMTPSQQKDLGITFNSTTGQFDVNGKAVIDGIAGSEMEVAIYDITNARTPKLVSKTYY

SEQ ID NO: 20 - DNA, 888 bp, Artificial Sequence
  Synthetic
atggcagagattgaggagaacaagaactccaccactgctctctcagagagcagggaggagctgtttggcattgtggatgtggacaagagctttgacaagatggctcaatattattatttcaaaaacctccaagggaagatagagaatggctacaagttcaccaactacaagggaagctcaaccaacctggacaagttcaagacaacctcagacaaggtttctgatcttcagatcactggcttgctgccagaatacattggtggcaacaccttcccaaaccacacagagcaagagcaaaccttctcaacaacaagctatgcaaggaccatcactgagagtgtgacaacaaccacccagagtggcttctctgtttctggccaaggaggcttgctgaacattcccatcatcttccccaatgggacaagcatcactgcttcaatcaacacaagttccaccttggccaagcaagagagtgacacaaggaccctcactgctccaagccaagctgtgaaggtgcctcctgggaagacctacagagcagaggtgaagctggtgaggaagagcttcaaggccaatgttggcttcaatggaaccattgctcctcctctctcaactgacatctgcaatgctggccacctctcccaatgtggagctggaggaaggcccaacaagactgtcaccaagagctatgccacccaaaccctctacagggacatgacaccctcccagcagaaggatttggggatcaccttcaacagcaccactggccagtttgatgtgaatgggaaggctgtgattgatggcattgctggctcagagatggaggtggccatctatgacatcaccaatgcaaggaccccaaagctggtgagcaagacatattactaa

SEQ ID NO: 21 - protein, 295 aa, Artificial Sequence; identical to SEQ ID NO: 19
  Synthetic

SEQ ID NO: 22 - DNA, 888 bp, Artificial Sequence
  Synthetic
atggcagagattgaggagaacaagaactccaccactgccctctcagagagcagggaggagctgtttggcattgtggatgtggacaagagcttcgacaagatggcccaatattattatttcaaaaacctccaagggaagattgagaatggctacaagttcaccaactacaaggggagcagcaccaacctggacaagttcaagaccacctcagacaaggtgagtgacctccaaatcaccgggctgctgccagagtacattgggggcaacaccttccccaaccacacagagcaggagcagaccttctcaaccaccagctatgccaggaccatcactgagagtgtcaccaccaccacccagagtggcttctctgtgagtggccagggagggctgctgaacattcccatcatcttccccaatggcaccagcatcaccgcctcaatcaacacaagttccaccctggccaagcaggagagtgacaccaggaccctcactgctccctcccaggctgtgaaggtgcctcctgggaagacctacagggcagaggtgaagctggtgaggaagagcttcaaggccaatgtgggcttcaatggcaccattgcccctcccctctcaactgacatctgcaatgctggccacctctcccagtgtggggctggggggaggcccaacaagaccgtcaccaagagctatgccacccagaccctctacagggacatgacaccctcccagcagaaggacttggggatcaccttcaacagcaccactggccagtttgatgtgaatgggaaggctgtgattgatggcattgctggctcagagatggaggtggccatctatgacatcaccaatgcaaggacccccaagctggtgagcaagacatattactaa

SEQ ID NO: 23 - protein, 295 aa, Artificial Sequence; identical to SEQ ID NO: 19
  Synthetic

SEQ ID NO: 24 - DNA, 909 bp, Artificial Sequence
  Synthetic
atggcggagattgaagaaaacaagaactccacaactgctctttcggaatcaagagaagagctctttggaattgttgatgtcgacaaaagtttcgacaagatggcccagtattactacttcaagaatctctgcaatgctggccatctctctcaatgccaagggaagatagagaacgggtacaaattcacgaactacaagggaagctcaacgaatctcgataagttcaaaacgacctccgataaagtctctgacttgcaaataaccggcttgcttcctgaatatattgggggaaacactttcccaaatcataccgagcaagagcaaaccttttcaaccacaagctacgcacgtacaataactgagagtgtcacgacaacgactcaaagcggattttctgtttctggacaaggaggattgttgaacatccccatcatttttccaaacggaacctcaatcactgcttcaatcaacacttcctcgactttggcaaagcaagagagtgatacaaggactttaactgcaccttctcaagctgtgaaggttccacctggaaaaacatatcgagctgaagtgaagctggtgagaaaatccttcaaagcgaatgtgggattcaatggaacaattgctcctcccctttcaactgacataaagatgaatgcaagatgggaaggagctggaggaaggccaaataaaactgtcacaaagagctatgccacacaaactctttatcgggatatgaccccttctcaacaaaaagatttggggatcaccttcaactcaacaactggccaatttgacgtgaatgggaaagccgttattgatggaattgctggaagcgaaatggaagtcgcaatctacgatattaccaatgctcgaactccgaagctcgtttcgaagacctattactaa

SEQ ID NO: 25 - protein, 302 aa, Artificial Sequence
  Synthetic
MAEIEENKNSTTALSESREELFGIVDVDKSFDKMAQYYYFKNLCNAGHLSQCQGKIENGYKFTNYKGSSTNLDKFKTTSDKVSDLQITGLLPEYIGGNTFPNHTEQEQTFSTTSYARTITESVTTTTQSGFSVSGQGGLLNIPIIFPNGTSITASINTSSTLAKQESDTRTLTAPSQAVKVPPGKTYRAEVKLVRKSFKANVGFNGTIAPPLSTDIKMNARWEGAGGRPNKTVTKSYATQTLYRDMTPSQQKDLGITFNSTTGQFDVNGKAVIDGIAGSEMEVAIYDITNARTPKLVSKTYY

SEQ ID NO: 26 - DNA, 909 bp, Artificial Sequence
  Synthetic
atggcagagattgaggagaacaagaactccaccactgctctctcagagagcagggaggagctgtttggcattgtggatgtggacaagagctttgacaagatggctcaatattattatttcaaaaacctctgcaatgctggccacttgagccaatgccaagggaagattgagaatggctacaagttcaccaactacaagggaagctcaaccaacctggacaagttcaagacaacctcagacaaggtttctgatctccagatcactggcttgctgccagaatacattggtggcaacaccttcccaaaccacacagagcaagagcaaaccttctcaacaacaagctatgcaaggaccatcactgagagtgtgacaacaaccacccagagtggcttctctgtttctggccaaggaggcttgctgaacattcccatcatcttccccaatgggacaagcatcactgcttcaatcaacacaagttccaccttggccaagcaagagagtgacacaaggaccctcactgctccaagccaagctgtgaaggtgcctcctgggaagacctacagagcagaggtgaagctggtgaggaagagcttcaaggccaatgttggcttcaatggaaccattgctcctcctctctcaactgacatcaagatgaatgcaaggtgggaaggagctggaggaaggcccaacaagactgtcaccaagagctatgccacccaaaccctctacagggacatgacaccctcccagcagaaggatttggggatcaccttcaacagcaccactggccagtttgatgtgaatgggaaggctgtgattgatggcattgctggctcagagatggaggtggccatctatgacatcaccaatgcaaggaccccaaagctggtgagcaagacatattactaa

SEQ ID NO: 27 - protein, 302 aa, Artificial Sequence; identical to SEQ ID NO: 25
  Synthetic

SEQ ID NO: 28 - DNA, 909 bp, Artificial Sequence
  Synthetic
atggcagagattgaggagaacaagaactccaccactgccctctcagagagcagggaggagctgtttggcattgtggatgtggacaagagcttcgacaagatggcccaatattattatttcaaaaacctctgcaatgctggccacctgagccagtgccaagggaagattgagaatggctacaagttcaccaactacaaggggagcagcaccaacctggacaagttcaagaccacctcagacaaggtgagtgacctccagatcaccgggctgctgccagagtacattgggggcaacaccttccccaaccacacagagcaggagcagaccttctcaaccaccagctatgccaggaccatcactgagagtgtcaccaccaccacccagagtggcttctctgtgagtggccagggagggctgctgaacattcccatcatcttccccaatggcaccagcatcactgcctcaatcaacacaagttccaccctggccaagcaggagagtgacaccaggaccctcactgctccctcccaggctgtgaaggtgcctcctgggaagacctacagggcagaggtgaagctggtgaggaagagcttcaaggccaatgtgggcttcaatggcaccattgcccctcccctctcaactgacatcaagatgaatgccaggtgggagggggctggggggaggcccaacaagaccgtcaccaagagctatgccacccagaccctctacagggacatgacaccctcccagcagaaggacttggggatcaccttcaacagcaccactggccagtttgatgtgaatgggaaggctgtgattgatggcattgctggctcagagatggaggtggccatctatgacatcaccaatgcaaggacccccaagctggtgagcaagacatattactaa

SEQ ID NO: 29 - protein, 302 aa, Artificial Sequence; identical to SEQ ID NO: 25
  Synthetic

SEQ ID NO: 30 - DNA, 888 bp, Artificial Sequence
  Synthetic
atggcggagattgaagaaaacaagaactccacaactgctctttcggaatcaagagaagagctctttggaattgttgatgtcgacaaaagtttcgacaagatggcccagtattactacttcaagaatctccaagggaagatagagaacgggtacaaattcacgaactacaagggaagctcaacgaatctcgataagttcaaaacgacctgcaatgctggccatctctctcaatgccaaataaccggcttgcttcctgaatatattgggggaaacactttcccaaatcataccgagcaagagcaaaccttttcaaccacaagctacgcacgtacaataactgagagtgtcacgacaacgactcaaagcggattttctgtttctggacaaggaggattgttgaacatccccatcatttttccaaacggaacctcaatcactgcttcaatcaacacttcctcgactttggcaaagcaagagagtgatacaaggactttaactgcaccttctcaagctgtgaaggttccacctggaaaaacatatcgagctgaagtgaagctggtgagaaaatccttcaaagcgaatgtgggattcaatggaacaattgctcctcccctttcaactgacataaagatgaatgcaagatgggaaggagctggaggaaggccaaataaaactgtcacaaagagctatgccacacaaactctttatcgggatatgaccccttctcaacaaaaagatttggggatcaccttcaactcaacaactggccaatttgacgtgaatgggaaagccgttattgatggaattgctggaagcgaaatggaagtcgcaatctacgatattaccaatgctcgaactccgaagctcgtttcgaagacctattactaa

SEQ ID NO: 31 - protein, 295 aa, Artificial Sequence
  Synthetic
MAEIEENKNSTTALSESREELFGIVDVDKSFDKMAQYYYFKNLQGKIENGYKFTNYKGSSTNLDKFKTTCNAGHLSQCQITGLLPEYIGGNTFPNHTEQEQTFSTTSYARTITESVTTTTQSGFSVSGQGGLLNIPIIFPNGTSITASINTSSTLAKQESDTRTLTAPSQAVKVPPGKTYRAEVKLVRKSFKANVGFNGTIAPPLSTDIKMNARWEGAGGRPNKTVTKSYATQTLYRDMTPSQQKDLGITFNSTTGQFDVNGKAVIDGIAGSEMEVAIYDITNARTPKLVSKTYY

SEQ ID NO: 32 - DNA, 888 bp, Artificial Sequence
  Synthetic
atggcagagattgaggagaacaagaactccaccactgctctctcagagagcagggaggagctgtttggcattgtggatgtggacaagagctttgacaagatggctcaatattattatttcaaaaacctccaagggaagatagagaatggctacaagttcaccaactacaagggaagctcaaccaacttggacaagttcaagaccacctgcaatgctggccacctctcccaatgccagatcactggcttgctgccagaatacattggtggcaacaccttcccaaaccacactgagcaagagcaaaccttctcaacaacaagctatgcaaggaccatcacagagagtgtgacaacaaccacccagagtggcttctctgtttctggccaaggaggcttgctgaacattcccatcatcttcccaaatgggacaagcatcactgcttcaatcaacacaagttccaccttggccaagcaagagagtgacacaaggaccctcactgctccaagccaagctgtgaaggtgcctcctgggaagacctacagagcagaggtgaagctggtgaggaagagcttcaaggccaatgttggcttcaatggaaccattgctcctcctctctcaactgacatcaagatgaatgcaaggtgggaaggagctggaggaaggcccaacaagactgtcaccaagagctatgccacccaaaccctctacagggacatgacaccaagccagcagaaggatttggggatcaccttcaacagcaccactggccagtttgatgtgaatgggaaggctgtgattgatggcattgctggctcagagatggaggtggccatctatgacatcaccaatgcaaggaccccaaagctggtgagcaagacatattactaa

SEQ ID NO: 33 - protein, 295 aa, Artificial Sequence; identical to SEQ ID NO: 31
  Synthetic

SEQ ID NO: 34 - DNA, 888 bp, Artificial Sequence
  Synthetic
atggcagagattgaggagaacaagaactccaccactgccctctcagagagcagggaggagctgtttggcattgtggatgtggacaagagctttgacaagatggcccaatattattatttcaaaaacctccaagggaagatagagaatggctacaagttcaccaactacaagggctcaagcaccaacctggacaagttcaagaccacctgcaatgctggccacctctcccagtgccagatcaccgggctgctgccagagtacattgggggcaacaccttccccaaccacactgagcaggagcagaccttctcaaccaccagctatgccaggaccatcaccgagagtgtgaccaccaccacccagagtggcttctctgtgagtggccagggagggctgctgaacattcccatcatcttccccaatggcaccagcatcactgcctcaatcaacacaagttccaccctggccaagcaggagagtgacaccaggaccctcactgctccctcccaggctgtgaaggtgcctcctgggaagacctacagggcagaggtgaagctggtgaggaagagcttcaaggccaatgtgggcttcaatggcaccattgcccctcccctctcaactgacatcaagatgaatgccaggtgggagggggctggggggaggcccaacaagaccgtcaccaagagctatgccacccagaccctctacagggacatgacaccctcccagcagaaggacttggggatcaccttcaacagcaccactggccagtttgatgtgaatgggaaggctgtgattgatggcattgctggctcagagatggaggtggccatctatgacatcaccaatgcaaggacccccaagctggtgagcaagacatattactaa

SEQ ID NO: 35 - protein, 295 aa, Artificial Sequence; identical to SEQ ID NO: 31
  Synthetic

SEQ ID NO: 36 - DNA, 888 bp, Artificial Sequence
  Synthetic
atggcggagattgaagaaaacaagaactccacaactgctctttcggaatcaagagaagagctctttggaattgttgatgtcgacaaaagtttcgacaagatggcccagtattactacttcaagaatctccaagggaagatagagaacgggtacaaattcacgaactacaagggaagctcaacgaatctcgataagttcaaaacgacctccgataaagtctctgacttgcaaataaccggcttgcttcctgaatatattgggggaaacactttcccaaatcataccgagcaagagcaaaccttttcaaccacaagctacgcacgtacaataactgagagtgtcacgacaacgactcaaagcggattttctgtttctggacaaggaggattgttgaacatccccatcatttttccaaacggaacctcaatcactgcttcaatcaacacttcctcgactttggcaaagcaagagagtgatacaaggactttaactgcaccttctcaagctgtgaaggttccacctggaaaaacatatcgagctgaagtgaagctggtgagaaaatccttcaaagcgaatgtgggattcaatggaacaattgctcctcccctttcaactgacataaagatgaatgcaagatgggaaggagctggaggaaggccaaataaaactgtcacaaagagctatgccacacaaactctttatcgggatatgaccccttctcaacaaaaagatttggggatcaccttcaactcaacaactggccaatttgacgtgaatgggaaagccgttattgatggaattgcttgcaatgctggccatctctctcaatgcatctacgatattaccaatgctcgaactccgaagctcgtttcgaagacctattactaa

SEQ ID NO: 37 - protein, 295 aa, Artificial Sequence
  Synthetic
MAEIEENKNSTTALSESREELFGIVDVDKSFDKMAQYYYFKNLQGKIENGYKFTNYKGSSTNLDKFKTTSDKVSDLQITGLLPEYIGGNTFPNHTEQEQTFSTTSYARTITESVTTTTQSGFSVSGQGGLLNIPIIFPNGTSITASINTSSTLAKQESDTRTLTAPSQAVKVPPGKTYRAEVKLVRKSFKANVGFNGTIAPPLSTDIKMNARWEGAGGRPNKTVTKSYATQTLYRDMTPSQQKDLGITFNSTTGQFDVNGKAVIDGIACNAGHLSQCIYDITNARTPKLVSKTYY

SEQ ID NO: 38 - DNA, 888 bp, Artificial Sequence
  Synthetic
atggcggagattgaagaaaacaagaactccacaactgctctttcggaatcaagagaagagctctttggaattgttgatgtcgacaaaagtttcgacaagatggcccagtattactacttcaagaatctccaagggaagatagagaacgggtacaaattcacgaactacaagggaagctcaacgaatctcgataagttcaaaacgacctccgataaagtctctgacttgcaaataaccggcttgcttcctgaatatattgggggaaacactttcccaaatcataccgagcaagagcaaaccttttcaaccacaagctacgcacgtacaataactgagagtgtcacgacaacgactcaaagcggattttctgtttctggacaaggaggattgttgaacatccccatcatttttccaaacggaacctcaatcactgcttcaatcaacacttcctcgactttggcaaagcaagagagtgatacaaggactttaactgcaccttctcaagctgtgaagtgcaatgctggccatctctctcaatgccgagctgaagtgaagctggtgagaaaatccttcaaagcgaatgtgggattcaatggaacaattgctcctcccctttcaactgacataaagatgaatgcaagatgggaaggagctggaggaaggccaaataaaactgtcacaaagagctatgccacacaaactctttatcgggatatgaccccttctcaacaaaaagatttggggatcaccttcaactcaacaactggccaatttgacgtgaatgggaaagccgttattgatggaattgctggaagcgaaatggaagtcgcaatctacgatattaccaatgctcgaactccgaagctcgtttcgaagacctattactaa

SEQ ID NO: 39 - protein, 295 aa, Artificial Sequence
  Synthetic
MAEIEENKNSTTALSESREELFGIVDVDKSFDKMAQYYYFKNLQGKIENGYKFTNYKGSSTNLDKFKTTSDKVSDLQITGLLPEYIGGNTFPNHTEQEQTFSTTSYARTITESVTTTTQSGFSVSGQGGLLNIPIIFPNGTSITASINTSSTLAKQESDTRTLTAPSQAVKCNAGHLSQCRAEVKLVRKSFKANVGFNGTIAPPLSTDIKMNARWEGAGGRPNKTVTKSYATQTLYRDMTPSQQKDLGITFNSTTGQFDVNGKAVIDGIAGSEMEVAIYDITNARTPKLVSKTYY

SEQ ID NO: 40 - DNA, 888 bp, Artificial Sequence
  Synthetic
atggcggagattgaagaaaacaagaactccacaactgctctttcggaatcaagagaagagctctttggaattgttgatgtcgacaaaagtttcgacaagatggcccagtattactacttcaagaatctccaagggaagatagagaacgggtacaaattcacgaactacaagggaagctcaacgaatctcgataagttcaaaacgacctccgataaagtctctgacttgcaaataaccggcttgcttcctgaatatattgggggaaacactttcccaaatcataccgagcaagagcaaaccttttcaaccacaagctacgcacgtacaataactgagagtgtcacgacaacgactcaaagcggattttctgtttctggacaaggaggattgttgaacatccccatcatttttccaaacggaacctcaatcactgcttcaatcaacacttcctcgactttggcaaagcaagagagtgatacaaggactttaactgcaccttctcaagctgtgaaggttccacctggaaaaacatatcgagctgaagtgaagctggtgagaaaatccttcaaagcgaatgtgggattcaatggaacaattgctcctcccctttcaactgacataaagatgaatgcaagatgggaaggagctggaggaaggccaaataaaacttgcaatgctggccatctctctcaatgccaaactctttatcgggatatgaccccttctcaacaaaaagatttggggatcaccttcaactcaacaactggccaatttgacgtgaatgggaaagccgttattgatggaattgctggaagcgaaatggaagtcgcaatctacgatattaccaatgctcgaactccgaagctcgtttcgaagacctattactaa

SEQ ID NO: 41 - protein, 295 aa, Artificial Sequence
  Synthetic
MAEIEENKNSTTALSESREELFGIVDVDKSFDKMAQYYYFKNLQGKIENGYKFTNYKGSSTNLDKFKTTSDKVSDLQITGLLPEYIGGNTFPNHTEQEQTFSTTSYARTITESVTTTTQSGFSVSGQGGLLNIPIIFPNGTSITASINTSSTLAKQESDTRTLTAPSQAVKVPPGKTYRAEVKLVRKSFKANVGFNGTIAPPLSTDIKMNARWEGAGGRPNKTCNAGHLSQCQTLYRDMTPSQQKDLGITFNSTTGQFDVNGKAVIDGIAGSEMEVAIYDITNARTPKLVSKTYY